(19) United States
(12) Patent Application Publication
(10) Pub. No.: US 2021/0127993 A1
(43) Pub. Date: May 6, 2021

(54) PULSE TRANSIT TIME MEASUREMENT DEVICE AND BLOOD PRESSURE MEASUREMENT DEVICE

(71) Applicants: OMRON HEALTHCARE Co., Ltd., Kyoto (JP); OMRON Corporation, Kyoto (JP)

(72) Inventors: Naomi MATSUMURA, Kyoto (JP); Yasuhiro KAWABATA, Kyoto (JP); Kenji FUJII, Kyoto (JP); Reiji FUJITA, Kyoto (JP); Akito ITO, Kyoto (JP)

(21) Appl. No.: 17/143,334

(22) Filed: Jan. 7, 2021

Related U.S. Application Data
(63) Continuation of application No. PCT/JP2019/026084, filed on Jul. 1, 2019.

(30) Foreign Application Priority Data
Jul. 12, 2018 (JP) 2018-132390

(51) Int. Cl. A61B 5/021; A61B 5/28; A61B 5/318

(52) U.S. Cl. CPC A61B 5/02125; A61B 5/28; A61B 2562/0247; A61B 5/318; A61B 5/02141

(57) ABSTRACT

A pulse transit time measurement device includes a belt unit wound around a target measurement site of a user, an electrode group provided in the belt unit and including a four electrodes, a current source applying an alternating current between the first electrode and the second electrode, a potential difference signal detection unit detecting a potential difference signal between the third electrode and the fourth electrode, an electrocardiogram acquisition unit acquiring, based on the potential difference signal, an electrocardiogram corresponding to a waveform signal representative of an electrical activity of a heart of the user, a pulse wave signal acquisition unit acquiring, based on the potential difference signal, as a pulse wave signal, a waveform signal representative of an electrical impedance in the target measurement site of the user, and a pulse transit time calculation unit calculating a pulse transit time based on the electrocardiogram and the pulse wave signal.

### CROSS-REFERENCE TO RELATED APPLICATIONS

This application is the U.S. national stage application filed pursuant to 35 U.S.C. 365(c) and 120 as a continuation of International Patent Application No. PCT/JP2019/026084, filed Jul. 1, 2019, which application claims priority from Japanese Patent Application No. 2018-132390, filed Jul. 12, 2018, which applications are incorporated herein by reference in their entireties.

### TECHNICAL FIELD

The present invention relates to a pulse transit time measurement device that non-invasively measures pulse transit time and a blood pressure measurement device using the pulse transit time measurement device.

### BACKGROUND ART

A method of measuring a pulse transit time (PTT) is available that includes: detecting a pulse wave at two points on the artery; and calculating the time required for the pulse wave to propagate a distance between two points as the pulse transit time. For an increased temporal resolution of the pulse transit time measurement, the distance between two points is desirably increased.

Patent Document 1 discloses a technique for measuring the pulse transit time by monitoring changes in bioelectrical impedance caused by the pulse wave at two sites of the upper arm and an intermediate portion between the elbow and the wrist.

### CITATION LIST

### Patent Literature

Patent Document 1: JP 4105378 B

### SUMMARY OF INVENTION

### Technical Problem

In the technology disclosed in Patent Document 1, electrodes need to be attached at each of four sites of the shoulder, the wrist, the upper arm, and the intermediate portion between the elbow and the wrist. Thus, in a case where measurement is made for a long period of time, the attachment of the electrodes places a heavy physical burden on the user.

The present invention has been made in view of the above circumstances, and an object of the present invention is to provide a pulse transit time measurement device and a blood pressure measurement device in which a physical burden on the user due to the attachment is reduced.

### Solution to Problem

The present invention adopts the following configurations in order to solve the above problems.

A pulse transit time measurement device according to a first aspect includes a belt unit wound around a target measurement site of a user, an electrode group provided in the belt unit and including a first electrode, a second electrode, a third electrode, and a fourth electrode, a current source applying an alternating current between the first electrode and the second electrode, a potential difference signal detection unit detecting a potential difference signal between the third electrode and the fourth electrode, an electrocardiogram acquisition unit acquiring, based on the potential difference signal, an electrocardiogram corresponding to a waveform signal representative of an electrical activity of a heart of the user, a pulse wave signal acquisition unit acquiring, based on the potential difference signal, as a pulse wave signal, a waveform signal representative of an electrical impedance in the target measurement site of the user, and a pulse transit time calculation unit calculating a pulse transit time based on the electrocardiogram and the pulse wave signal.

According to the configuration described above, when the belt unit is wound around the target measurement site of the user, the electrode group is mounted on the user. This allows the user to measure the pulse transit time simply by installing one device on the user. Consequently, the attachment on the user is easy, and a physical burden placed on the user due to the attachment of the device is reduced. Furthermore, a circuit acquiring an electrocardiogram (ECG sensor) and a circuit acquiring a pulse wave signal (pulse wave sensor) share the third electrode, the fourth electrode, and the potential difference signal detection unit. Thus, the belt unit can be made smaller and part costs can be reduced.

In the first aspect, the electrode group may include a plurality of the third electrodes, and the plurality of third electrodes are arranged in one direction. In this case, the pulse transit time measurement device further includes a first switch circuit switching, among the plurality of third electrodes. to the third electrode to be connected to the potential difference signal detection unit.

According to the above-described configuration, an electrocardiogram and a pulse wave signal having a higher signal-to-noise ratio can be acquired. As a result, measurement accuracy for the pulse transit time can be improved.

In the first aspect, the electrode group may include a plurality of the fourth electrodes, and the plurality of fourth electrodes are arranged in the one direction. In this case, the pulse transit time measurement device further includes a second switch circuit switching, among the plurality of fourth electrodes, to the fourth electrode to be connected to the potential difference signal detection unit.

According to the above-described configuration, an electrocardiogram and a pulse wave signal having a higher signal-to-noise ratio can be acquired. As a result, measurement accuracy for the pulse transit time can be improved.

A pulse transit time measurement device according to a second aspect includes a belt unit wound around a target measurement site of a user, an electrode group wound around the belt unit and including a first electrode, a second electrode, a plurality of third electrodes arranged in a row, and a fourth electrode, a current source applying an alternating current between the first electrode and the second electrode, a first potential difference signal detection unit detecting a first potential difference signal corresponding to a potential difference signal between one of the plurality of third electrodes and the fourth electrode, a pulse wave signal acquisition unit acquiring, based on the first potential difference signal, as a pulse wave signal, a waveform signal representative of an electrical impedance in the target measurement site of the user, a second potential difference signal detection unit detecting a second potential difference signal corresponding to a potential difference signal between two of the third electrodes selected from among the plurality of third electrodes, an electrocardiogram acquisition unit acquiring, based on the second potential difference signal, an electrocardiogram corresponding to a waveform signal representative of an electrical activity of a heart of the user, and a pulse transit time calculation unit calculating a pulse transit time based on the electrocardiogram and the pulse signal.

According to the configuration described above, identical effects to those described with respect to the pulse transit time measurement device according to the first aspect are obtained.

A blood pressure measurement device according to a third aspect includes the above-described pulse transit time measurement device and a first blood pressure value calculation unit calculating a first blood pressure value based on the pulse transit time that is calculated.

According to the above-described configuration, blood pressure can be continuously measured over an extended period of time with a reduced physical burden on the user.

In the third aspect, the blood pressure measurement device may further include a pressing cuff provided in the belt unit, a fluid supply unit supplying a fluid to the pressing cuff, a pressure sensor detecting a pressure in the pressing cuff, and a second blood pressure value calculation unit calculating a second blood pressure value based on an output of the pressure sensor.

According to the configuration described above, continuous blood pressure measurement (blood pressure measurement based on the pulse transit time) and blood pressure measurement using an oscillometric method can be executed with one device. As a result, the configuration is highly convenient to the user.

### Advantageous Effects of Invention

According to the present invention, a pulse transit time measurement device and a blood pressure measurement device in which a physical burden on the user due to the attachment is reduced.

### BRIEF DESCRIPTION OF DRAWINGS

FIG. 1 is a diagram illustrating a blood pressure measurement device according to an embodiment.

FIG. 2 is a diagram illustrating the appearance of the blood pressure measurement device illustrated in FIG. 1.

FIG. 3 is a diagram illustrating the appearance of the blood pressure measurement device illustrated in FIG. 1.

FIG. 4 is a cross-sectional diagram illustrating the blood pressure measurement device illustrated in FIG. 1.

FIG. 5 is a block diagram illustrating a hardware configuration of a control system of the blood pressure measurement device illustrated in FIG. 1.

FIG. 6 is a block diagram illustrating a software configuration of the blood pressure measurement device illustrated in FIG. 1.

FIG. 7 is a diagram illustrating a method in which a pulse transit time calculation unit illustrated in FIG. 6 calculates a pulse transit time.

FIG. 8 is a flowchart illustrating operation in which the blood pressure measurement device illustrated in FIG. 1 performs blood pressure measurement based on the pulse transit time.

FIG. 9 is a flowchart illustrating operation in which the blood pressure measurement device illustrated in FIG. 1 performs blood pressure measurement using an oscillometric method.

FIG. 10 is a diagram illustrating changes in cuff pressure and pulse wave signal in blood pressure measurement using the oscillometric method.

FIG. 11 is a flowchart illustrating a method of adjusting a contact state between an electrode and an upper arm using a pressing cuff according to an embodiment.

FIG. 12 is a diagram illustrating the appearance of a blood pressure measurement device according to an embodiment.

FIG. 13 is a diagram illustrating the appearance of a blood pressure measurement device according to an embodiment.

FIG. 14 is a block diagram illustrating a hardware configuration of a control system of the blood pressure measurement device illustrated in FIG. 13.

FIG. 15 is a flowchart illustrating a method of selecting a detection electrode pair used to acquire a pulse wave signal and an electrocardiogram, according to an embodiment.

FIG. 16 is a diagram illustrating the appearance of a blood pressure measurement device according to an embodiment.

FIG. 17 is a diagram illustrating the appearance of a blood pressure measurement device according to an embodiment.

FIG. 18 is a block diagram illustrating a hardware configuration of a control system of the blood pressure measurement device illustrated in FIG. 17.

### DESCRIPTION OF EMBODIMENTS

Hereinafter, an embodiment of the present invention will be described with reference to the drawings.

### APPLICATION EXAMPLE

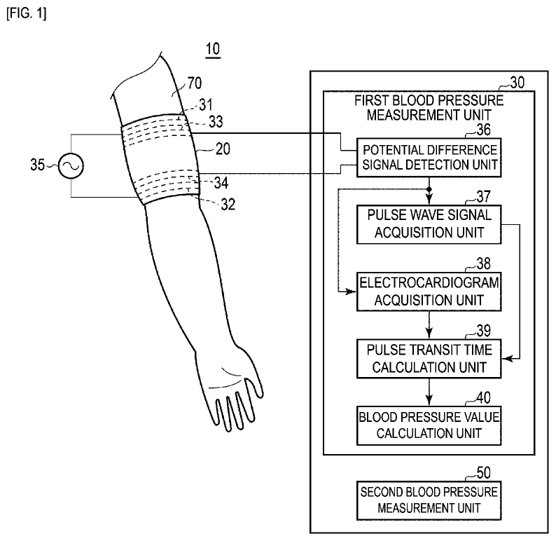

With reference to FIG. 1, an example of a case to which the present invention is applied will be described. FIG. 1 illustrates a blood pressure measurement device 10 according to an embodiment. The blood pressure measurement device 10 is a wearable device and is worn on an upper arm 70 of a user as a target measurement site. The blood pressure measurement device 10 includes a belt unit 20, a first blood pressure measurement unit 30, and a second blood pressure measurement unit 50.

The belt unit 20 is a member that is wound around the upper arm 70 of the user and is used to attach the blood pressure measurement device 10 to the upper arm 70 of the user.

The first blood pressure measurement unit 30 and the second blood pressure measurement unit 50 are provided in the belt unit 20. The first blood pressure measurement unit 30 non-invasively measures a pulse transit time and calculates a blood pressure value based on the measured pulse transit time. The first blood pressure measurement unit 30 can perform continuous blood pressure measurement for obtaining the blood pressure value for each beat. The second blood pressure measurement unit 50 performs blood pressure measurement using a method different from that of the first blood pressure measurement unit 30. The second blood pressure measurement unit 50 is based on, for example, an oscillometric method or a Korotkoff method and performs blood pressure measurement at a specific timing, for example, in response to operation performed by the user. The second blood pressure measurement unit 50 can measure the blood pressure more accurately than the first blood pressure measurement unit 30.

The first blood pressure measurement unit 30 includes current electrodes 31 and 32, detection electrodes 33 and 34, a current source 35, a potential difference signal detection unit 36, a pulse wave signal acquisition unit 37, an electrocardiogram acquisition unit 38, a pulse transit time calculation unit 39, and a blood pressure value calculation unit 40.

The current electrodes 31 and 32 and the detection electrodes 33 and 34 are disposed on the inner circumferential surface of the belt unit 20 such that the blood pressure measurement device 10 is in contact with the upper arm 70 of the user in a state in which the blood pressure measurement device 10 is attached to the upper arm 70 of the user (hereinafter simply referred to as the “attachment state”). The inner circumferential surface of the belt unit 20 is a portion of the surface of the belt unit 20 that faces the upper arm 70 of the user in the attachment state. In the attachment state, the current electrodes 31 and 32 and the detection electrodes 33 and 34 are not externally visible, but in FIG. 1, the current electrodes 31 and 32 and the detection electrodes 33 and 34 are illustrated for purposes of description. The detection electrodes 33 and 34 are disposed between the current electrodes 31 and 32. More specifically, the current electrode 31, the detection electrode 33, the detection electrode 34, and the current electrode 32 are arranged in this order in the width direction of the belt unit 20. The width direction of the belt unit 20 corresponds to a direction along the upper arm artery passing through the upper arm 70 in the attachment state. The current electrodes 31 and 32 correspond to a first electrode and a second electrode of the present invention, and the detection electrodes 33 and 34 correspond to a third electrode and a fourth electrode of the present invention.

The current electrodes 31 and 32 are connected to the current source 35, and the current source 35 applies an alternating current between the current electrodes 31 and 32. The alternating current is applied to acquire a pulse wave signal as described below. The alternating current is, for example, a sinusoidal current. The detection electrodes 33 and 34 are connected to a potential difference signal detection unit 36, and the potential difference signal detection unit 36 detects a potential difference signal between the detection electrodes 33 and 34. The potential difference signal is output to the electrocardiogram acquisition unit 38 and the pulse wave signal acquisition unit 37.

Based on the potential difference signal received from the potential difference signal detection unit 36, the pulse wave signal acquisition unit 37 acquires, as a pulse wave signal, a waveform signal representative of the bioelectrical impedance in the upper arm 70 of the user. The bioelectrical impedance in the upper arm 70 of the user varies with the blood flow of the upper arm artery. Thus, the waveform signal representing the bioelectrical impedance in the upper arm 70 of the user indirectly represents the volume pulse wave in the upper arm 70 of the user. The waveform signal representing the impedance is not limited to a signal directly representative of the impedance and may be a signal indirectly representative of the impedance, such as a drop voltage observed in a case where an alternating current is passed through the upper arm 70. In the present embodiment, the current electrodes 31 and 32, the detection electrodes 33 and 34, the current source 35, the potential difference signal detection unit 36, and the pulse wave signal acquisition unit 37 are collectively referred to as a pulse wave sensor.

The electrocardiogram acquisition unit 38 acquires an ElectroCardioGram (ECG) of the user based on the potential difference signal received from the potential difference signal detection unit 36. Then electrocardiogram is a waveform signal that represents an electrical activity of the heart of the user. In the present embodiment, the detection electrodes 33 and 34, the potential difference signal detection unit 36, and the electrocardiogram acquisition unit 38 are collectively referred to as an ElectroCardioGraphic (ECG) sensor.

The pulse transit time calculation unit 39 receives a pulse wave signal from the pulse wave signal acquisition unit 37 and receives an electrocardiogram from the electrocardiogram acquisition unit 38. The pulse transit time calculation unit 39 calculates a pulse transit time based on a time difference between a waveform feature point in the electrocardiogram and a waveform feature point in the pulse wave signal. For example, the pulse transit time calculation unit 39 calculates the time difference between the waveform feature point in the electrocardiogram and the waveform feature point in the pulse wave signal and outputs the calculated time difference as the pulse transit time. The waveform feature point in the electrocardiogram is, for example, a peak point corresponding to an R wave, and the waveform feature point in the pulse wave signals is, for example, a rising point. In the present embodiment, the pulse transit time corresponds to a time required for a pulse wave to propagate through the artery from the heart to the upper arm. Thus, compared to a case where the pulse transit time between two points on the upper arm 70 is measured, the present embodiment improves temporal resolution.

The blood pressure value calculation unit 40 calculates a blood pressure value based on the pulse transit time calculated by the pulse transit time calculation unit 39 and on a blood pressure calculation formula. The blood pressure calculation formula is a relational formula that represents a correlation between the pulse transit time and the blood pressure. An example of a blood pressure calculation formula is illustrated below.

SBP=A₁/PTT²+A₂ (1)

Here, SBP represents systolic blood pressure, PTT represents the pulse transit time, and A₁ and A₂ are parameters.

The pulse transit time calculation unit 39 can calculate the pulse transit time for each beat, and thus the blood pressure value calculation unit 40 can calculate the blood pressure value for each beat.

As described above, in the present embodiment, the ECG sensor and the pulse wave sensor are both provided in the belt unit 20. This allows both the ECG sensor and the pulse wave sensor to be mounted on the user simply by winding the belt unit 20 around the upper arm. Thus, the device can be easily attached to the user, and the physical burden on the user (also referred to as the attachment burden) caused by the attachment of the blood pressure measurement device 10 can be reduced.

Furthermore, the ECG sensor and the pulse wave sensor share the detection electrodes 33 and 34 and the potential difference signal detection unit 36. This allows the blood pressure measurement device 10 to be made smaller, and furthermore, part costs can be reduced. The miniaturization of the blood pressure measurement device 10 contributes to reducing the attachment burden.

Hereinafter, the blood pressure measurement device 10 will be described more specifically.

### CONFIGURATION EXAMPLE

### Hardware Configuration

An example of a hardware configuration of the blood pressure measurement device 10 according to the present embodiment will be described with reference to FIGS. 2 to 6.

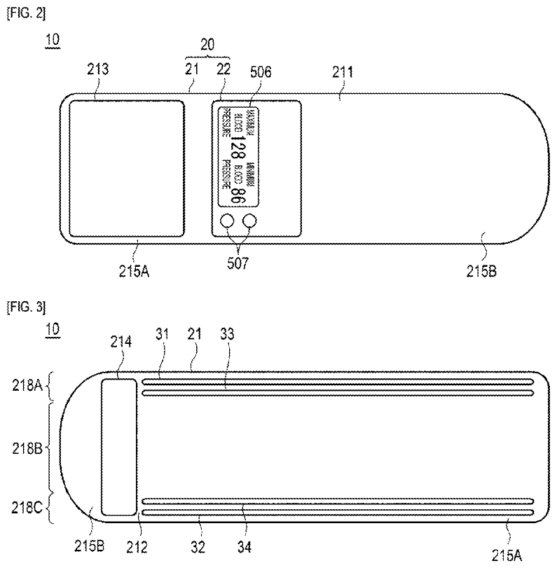

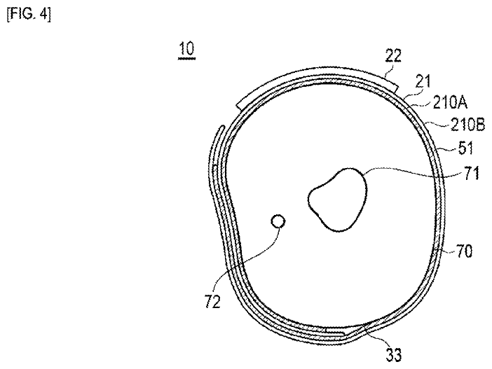

FIGS. 2 and 3 are plan views illustrating the appearance of the blood pressure measurement device 10. Specifically, FIG. 2 illustrates the blood pressure measurement device 10 viewed from the outer circumferential surface side of the belt unit 20, and FIG. 3 illustrates the blood pressure measurement device 10 viewed from the inner circumferential surface side of the belt unit 20. FIG. 4 illustrates a cross-section of the blood pressure measurement device 10 in the attachment state.

As illustrated in FIG. 2, the belt unit 20 includes a belt 21 and a body 22. The belt 21 is a belt-shaped member that is attached surrounding the upper arm 70 and may also be referred to by another name such as a “band” or “cuff ” The belt 21 has an outer circumferential surface 211 and an inner circumferential surface 212. The inner circumferential surface 212 is a surface facing the upper arm 70 of the user in the attachment state, and the outer circumferential surface 211 is a surface opposite to the inner circumferential surface 212.

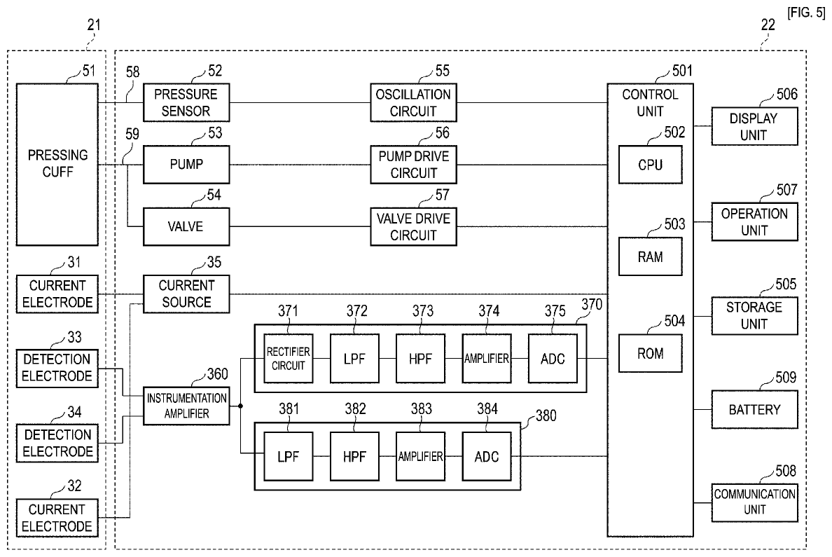

The body 22 is mounted on the belt 21. The body 22, together with a display unit 506 and an operation unit 507, houses components such as a control unit 501 (illustrated in FIG. 5) described below. The display unit 506 includes a display device displaying information such as a blood pressure measurement result. The display device may be, for example, a liquid crystal display (LCD), an organic Electro-Luminescence (EL) display, or the like. The organic EL display is sometimes referred to as an Organic Light Emitting Diode (OLED) display. The operation unit 507 is an input device that allows a user to input an instruction to the blood pressure measurement device 10. In an example in FIG. 2, the operation unit 507 includes, for example, a plurality of push type buttons. A touch screen that also serves as a display device and an input device may be used. The body 22 may be provided with a sound emitter such as a speaker or a piezoelectric sounder. The body 22 may be provided with a microphone to allow the user to input instructions by sounds.

The belt 21 includes an attachment member allowing the belt unit 20 to be attached to and detached from the upper arm. In the example illustrated in FIGS. 2 and 3, the attachment member is a surface fastener having: a loop surface 213 having a multiplicity of loops; and a hook surface 214 having a plurality of hooks. The loop surface 213 is disposed on the outer circumferential surface 211 of the belt 21 at a longitudinal end portion 215A of the belt 21. The longitudinal direction corresponds to the circumferential direction of the upper arm in the attachment state. The hook surface 214 is disposed on the inner circumferential surface 212 of the belt 21 at a longitudinal end portion 215B of the belt 21. The end portion 215B faces the end portion 215A in the longitudinal direction of the belt 21. When the loop surface 213 and the hook surface 214 are pressed against each other, the loop surface 213 and the hook surface 214 are joined. In addition, pulling the loop surface 213 and the hook surface 214 away from each other separates the loop surface 213 and the hook surface 214.

As illustrated in FIG. 3, current electrodes 31 and 32 and detection electrodes 33 and 34 are disposed on the inner circumferential surface 212 of the belt 21. The current electrodes 31 and 32 and the detection electrodes 33 and 34 each have an elongated shape in the longitudinal direction of the belt 21. In the blood pressure measurement device 10, a range of the available upper arm circumferential length is set. For example, the blood pressure measurement device 10 can be used for users with an upper arm circumferential length ranging from 220 to 320 mm. The dimensions of the current electrodes 31 and 32 and the detection electrodes 33 and 34 in the longitudinal direction of the belt 21 are equal to an upper limit value (for example, 320 mm) for the upper arm circumferential length. In this case, for any user who can use the blood pressure measurement device 10, the current electrodes 31 and 32 and the detection electrodes 33 and 34 encircle the upper arm 70 throughout the circumference.

Note that the dimension of each of the electrodes (for example, the detection electrode 33) in the longitudinal direction of the belt 21 may be a value at which the electrode surrounds a part of the upper arm 70. In one example, the electrode has a length that is half the upper limit value for the upper arm circumferential length (e.g., 160 mm). In other examples, the electrode has a length that is three quarters of the upper limit value for the upper arm circumferential length (e.g., 240 mm).

In addition, the dimension of the current electrodes 31 and 32 in the longitudinal direction of the belt 21 may be the same as the dimension of the detection electrodes 33 and 34 in the longitudinal direction, or may be longer or shorter than the dimension of the detection electrodes 33 and 34.

The current electrode 31 and the detection electrode 33 are disposed at a central end portion 218A of the belt 21. The central end portion 218A of the belt 21 is an end portion of the belt 21 in the width direction of the belt 21 and is an end portion located on the central side (shoulder side) in the attachment state. The width of the central end portion 218A is, for example, a quarter of the overall width of the belt 21. The current electrode 31 is located on the central side more than the detection electrode 33.

The current electrode 32 and the detection electrode 34 are disposed at a peripheral end portion 218C of the belt 21. The peripheral end portion 218C of the belt 21 is an end portion of the belt 21 in the width direction of the belt 21 and is an end portion located on the peripheral side (elbow side) in the attachment state. The width of the peripheral end portion 218C is, for example, a quarter of the overall width of the belt 21. The current electrode 32 is located on the peripheral side more than the detection electrode 34.

As illustrated in FIG. 4, the belt 21 includes an inner cloth 210A, an outer cloth 210B, and a pressing cuff 51 provided between the inner cloth 210A and the outer cloth 210B. The pressing cuff 51 is a longitudinally long band of the belt 21 such that the pressing cuff 51 can surround the upper arm 70. In the width direction of the belt 21, the pressing cuff 51 is present across the central end portion 218A, an intermediate portion 218B, and the peripheral end portion 218C. The intermediate portion 218B is a portion between the central end portion 218A and the peripheral end portion 218C. The pressing cuff 51 is used for blood pressure measurement using the oscillometric method. In a case where a structure such as an electrode is disposed in the intermediate portion 218B, the accuracy of the blood pressure measurement using the oscillometric method may be reduced. Thus, in the present embodiment, the current electrode 31 and the detection electrode 33 are disposed at the central end portion 218A of the belt 21, and the current electrode 32 and the detection electrode 34 are disposed at the peripheral end portion 218C of the belt 21. For example, the pressing cuff 51 is configured as a fluid bag by placing two stretchable polyurethane sheets opposite each other in the thickness direction and welding the edge portions of the polyurethane sheets.

FIG. 5 illustrates one example of the hardware configuration of a control system of the blood pressure measurement device 10. In the example of FIG. 5, in addition to the display unit 506 and the operating unit 507 described above, the body 22 houses the control unit 501, a storage unit 505, a communication unit 508, a battery 509, the current source 35, an instrumentation amplifier 360, a detection circuit 370, a detection circuit 380, a pressure sensor 52, a pump 53, a valve 54, an oscillator circuit 55, a pump drive circuit 56, and a valve drive circuit 57.

The control unit 501 includes a Central Processing Unit (CPU) 502, a Random Access Memory (RAM) 503, a Read Only Memory (ROM) 504, and the like and controls each component according to information processing. The storage unit 505 is, for example, an auxiliary storage device, for example, a hard disk drive (HDD) or a semiconductor memory (for example, a flash memory) and non-transitorily stores programs executed by the control unit 501 (including, for example, a pulse transit time measurement program and a blood pressure measurement program), settings data necessary for executing the programs, results of blood pressure measurement, and the like. A storage medium included in the storage unit 505 is, to enable computers, other devices, machines, or the like to read information such as recorded programs, a medium that stores information such as the programs, by using electrical, magnetic, optical, mechanical, or chemical actions. Note that some or all of the programs may be stored in the ROM 504.

The communication unit 508 is a communication interface for communicating with an external device such as a portable terminal of the user (for example, a smartphone). The communication unit 508 includes a wired communication module and/or a wireless communication module. As a wireless system, for example, Bluetooth (trade name), Bluetooth Low Energy (BLE), or the like can be adopted.

The battery 509 supplies power to components such as the control unit 501. The battery 509 is, for example, a rechargeable battery.

The current source 35 is connected to the current electrodes 31 and 32 and passes a high frequency constant current between the current electrodes 31 and 32. In this example, the current has a frequency of 50 kHz and a current value of 1 mA.

The instrumentation amplifier 360 is one example of the potential difference signal detection unit 36 illustrated in FIG. 1. The detection electrodes 33 and 34 are respectively connected to two input terminals of the instrumentation amplifier 360.

The instrumentation amplifier 360 differentially amplifies the potential of the detection electrode 33 and the potential of the detection electrode 34. The instrumentation amplifier 360 outputs a potential difference signal obtained by amplifying the potential difference between the detection electrode 33 and the detection electrode 34. The potential difference signal is branched into two signals, which are provided to the detection circuits 370 and 380.

The detection circuit 370 corresponds to the pulse wave signal acquisition unit 37 illustrated in FIG. 1. The detection circuit 370 extracts, from the potential difference signal, a signal component corresponding to the electrical impedance between the detection electrodes 33 and 34. In the example illustrated in FIG. 5, the detection circuit 370 includes a rectifier circuit 371, a low pass filter (LPF) 372, a high pass filter (HPF) 373, an amplifier 374, and an analog-to-digital converter (ADC) 375. In detection circuit 370, the potential difference signal is rectified by the rectifier circuit 371, filtered by the LPF 372, filtered by the HPF 373, amplified by the amplifier 374, and converted into a digital signal by ADC 375. The LPF 372 has a cutoff frequency of 10 Hz, for example, and the HPF 373 has a cutoff frequency of 0.5 Hz, for example. The control unit 501 acquires, as a pulse wave signal, a time series of potential difference signals output from the detection circuit 370.

The detection circuit 380 corresponds to the electrocardiogram acquisition unit 38 illustrated in FIG. 1. The detection circuit 380 extracts, from the potential difference signal, signal components corresponding to the electrical activity of the heart. In the example illustrated in FIG. 5, the detection circuit 380 includes an LPF 381, an HPF 382, an amplifier 383, and an ADC 384. In the detection circuit 380, the potential difference signal is filtered by the LPF 381, filtered by the HPF 382, amplified by the amplifier 383, and converted into a digital signal by the ADC 384. The LPF 381 has a cutoff frequency of 40 Hz, for example, and the HPF 382 has a cutoff frequency of 0.5 Hz, for example. The control unit 501 acquires, as an electrocardiogram, a time series of potential difference signals output from the detection circuit 380.

In the example illustrated in FIG. 5, the current electrodes 31 and 32, the detection electrodes 33 and 34, the current source 35, the instrumentation amplifier 360, the detection circuit 370, and the detection circuit 380 are included in the first blood pressure measurement unit 30 illustrated in FIG. 1.

The pressure sensor 52 is connected to the pressing cuff 51 via a pipe 58, and the pump 53 and the valve 54 are connected to the pressing cuff 51 via a pipe 59. The pipes 58 and 59 may be a single common pipe. The pump 53 is, for example, a piezoelectric pump and feeds air as a fluid to the pressing cuff 51 through the pipe 59, in order to increase a pressure inside the pressing cuff 51. The pump drive circuit 56 drives the pump 53 based on a control signal received from the control unit 501. The valve drive circuit 57 drives the valve 54 based on a control signal received from the control unit 501. When the valve 54 is in an open state, the pressing cuff 51 is in communication with the atmosphere and air in the pressing cuff 51 is discharged into the atmosphere.

The pressure sensor 52 detects the pressure in the pressing cuff 51 (also referred to as cuff pressure) and generates an electrical signal representative of the cuff pressure. The cuff pressure is, for example, a pressure based on the atmospheric pressure as a reference. The pressure sensor 52 is, for example, a piezoresistive pressure sensor. The oscillation circuit 55 oscillates based on the electric signal from the pressure sensor 52 and outputs, to the control unit 501, a frequency signal having a frequency in accordance with the electric signal. In this example, the output of the pressure sensor 52 is used to control the pressure of the pressing cuff 51 and to calculate the blood pressure value using the oscillometric method.

In the example illustrated in FIG. 5, the pressing cuff 51, the pressure sensor 52, the pump 53, the valve 54, the oscillation circuit 55, the pump drive circuit 56, the valve drive circuit 57, and the pipes 58 and 59 are included in the second blood pressure measurement unit 50 illustrated in FIG. 1.

Also, with respect to a specific hardware configuration of the blood pressure measurement device 10, components can be omitted, replaced, or added as appropriate in accordance with embodiments. For example, the control unit 501 may include a plurality of processors. The signal processing (e.g. filtering) executed on the potential difference signal may be digital signal processing.

### Software Configuration

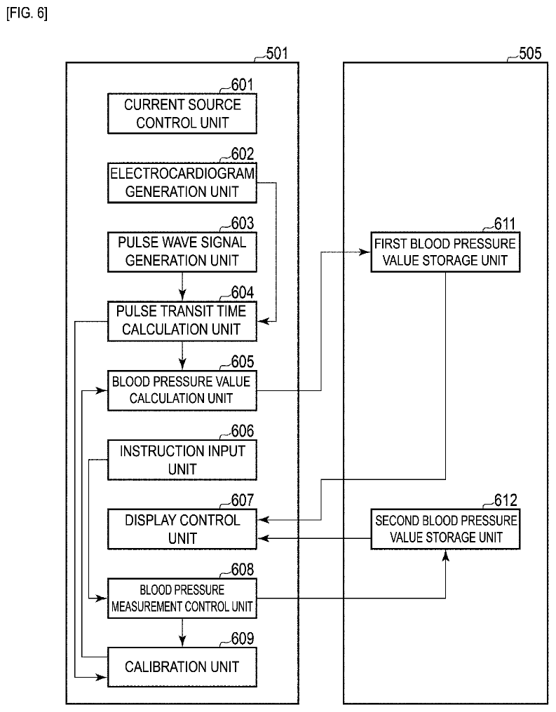

An example of a software configuration of the blood pressure measurement device 10 according to the present embodiment will be described with reference to FIG. 6. FIG. 6 illustrates one example of the software configuration of the blood pressure measurement device 10. In the example in FIG. 6, the blood pressure measurement device 10 includes a current source control unit 601, an electrocardiogram generation unit 602, a pulse wave signal generation unit 603, a pulse transit time calculation unit 604, a blood pressure value calculation unit 605, an instruction input unit 606, a display control unit 607, a blood pressure measurement control unit 608, a calibration unit 609, a first blood pressure value storage unit 611, and a second blood pressure value storage unit 612. The current source control unit 601, the electrocardiogram generation unit 602, the pulse wave signal generation unit 603, the pulse transit time calculation unit 604, the blood pressure value calculation unit 605, the instruction input unit 606, the display control unit 607, the blood pressure measurement control unit 608, and the calibration unit 609 execute the following processing in a case where the control unit 501 of the blood pressure measurement device 10 executes programs stored in the storage unit 505. When the control unit 501 executes the program, the control unit 501 loads the program in the RAM 503. Then, the control unit 501 causes the CPU 502 to interpret and execute the program loaded in the RAM 503 to control each component. The first blood pressure value storage unit 611 and the second blood pressure value storage unit 612 are implemented by the storage unit 505.

The current source control unit 601 controls the current source 35 to acquire the pulse wave signal. The current source control unit 601 provides the current source 35 with a drive signal driving the current source 35. When driven by the current source control unit 601, the current source 35 generates a high frequency current that is passed between the current electrodes 31 and 32.

The electrocardiogram generation unit 602 generates an electrocardiogram based on the output of the detection circuit 380. Specifically, the electrocardiogram generation unit 602 acquires, as an electrocardiogram, a time series of potential difference signals output from the detection circuit 380. The pulse wave signal generation unit 603 generates a pulse wave signal based on the output of the detection circuit 370. Specifically, the pulse wave signal generation unit 603 acquires, as a pulse wave signal, a time series of potential difference signals output from the detection circuit 370.

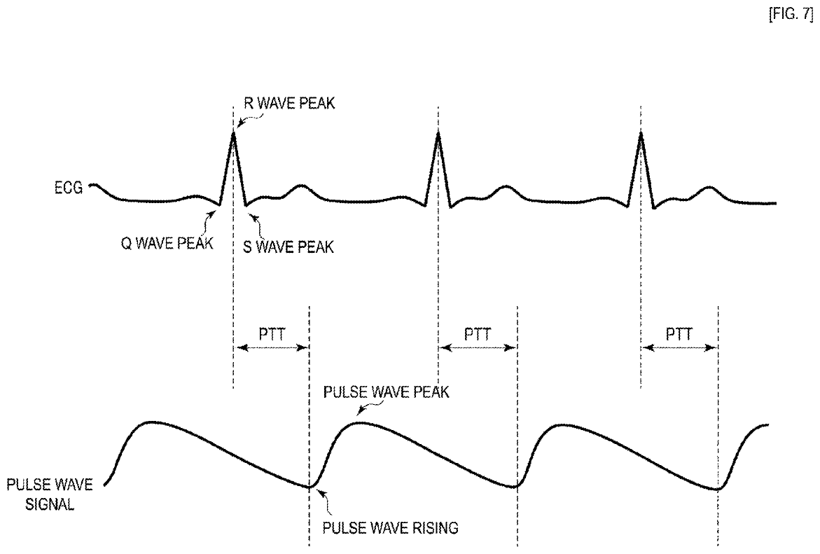

The pulse transit time calculation unit 604 receives an electrocardiogram from the electrocardiogram generation unit 602, receives a pulse wave signal from the pulse wave signal generation unit 603, and calculates a pulse transit time based on a time difference between a waveform feature point in the electrocardiogram and a waveform feature point in the pulse wave signal. For example, as illustrated in FIG. 7, the pulse transit time calculation unit 604 detects the time (point in time) of a peak point corresponding to an R wave in the electrocardiogram, detects the time (point in time) of a rising point in the pulse wave signal, and subtracts the time of the peak point from the time of the rising point to calculate the difference as the pulse transit time.

Note that the pulse transit time calculation unit 604 may correct the above-described time difference based on a PreEj ection Period (PEP) and output the corrected time difference as the pulse transit time. For example, with the preejection period considered to be constant, the pulse transit time calculation unit 604 may calculate the pulse transit time by subtracting a predetermined value from the time difference described above.

The peak point corresponding to the R wave is an example of a waveform feature point in the electrocardiogram. The waveform feature point in the electrocardiogram may be a peak point corresponding to a Q wave or a peak point corresponding to an S wave. Since the R wave appears as a distinct peak compared to the Q or S wave, the time of the R wave peak point can be more accurately identified. Thus, preferably, the R wave peak point is used as the waveform feature point in the electrocardiogram. Additionally, the rising point is an example of a waveform feature point in the pulse wave signal. The waveform feature point in the pulse wave signal may be the peak point.

With reference back to FIG. 6, the blood pressure value calculation unit 605 calculates a blood pressure value based on the pulse transit time calculated by the pulse transit time calculation unit 604 and on the blood pressure calculation formula. The blood pressure value calculation unit 605 uses Formula (1) above as a blood pressure calculation formula, for example. The blood pressure value calculation unit 605 causes the first blood pressure value storage section 611 to store the calculated blood pressure value in association with time information.

Note that the blood pressure calculation formula is not limited to Formula (1) above. The blood pressure calculation formula may be, for example, the following formula.

SBP=B₁/PTT²+B₂/PTT+B₃×PTT+B₄ (2)

Here, B₁, B₂, B₃, and B₄ are parameters.

The instruction input unit 606 receives an instruction input from the user through the operation unit 507. The instruction can be, for example, initiation of oscillometric blood pressure measurement, initiation of continuous blood pressure measurement (blood pressure measurement based on the pulse transit time), stoppage of the continuous blood pressure measurement, switching of display, etc. For example, when operation instructing initiation of blood pressure measurement is performed, the instruction input unit 606 provides the blood pressure measurement control unit 608 with an instruction signal instructing the execution of the blood pressure measurement using the oscillometric method.

The display control unit 607 controls the display unit 506. For example, the display control unit 607 causes the display unit 506 to display information such as results of the blood pressure measurement using the oscillometric method; and results of the continuous blood pressure measurement.

The blood pressure measurement control unit 608 controls the pump drive circuit 56 and the valve drive circuit 57 to execute the blood pressure measurement using the oscillometric method. When receiving the instruction signal from the instruction input unit 606, the blood pressure measurement control unit 608 brings the valve 54 to a closed state via the valve drive circuit 57 and drives the pump 53 via the pump drive circuit 56. This initiates supply of air to the pressing cuff 51. The pressing cuff 51 is inflated to compress the upper arm 70 of the user. The blood pressure measurement control unit 608 monitors the cuff pressure using the pressure sensor 52. The blood pressure measurement control unit 608 calculates the blood pressure value using the oscillometric method, based on a pressure signal output from the pressure sensor 52 in a pressurizing process in which air is fed to the pressing cuff 51. Although the blood pressure value includes the systolic blood pressure (SBP) and the diastolic blood pressure (DBP), it is not limited thereto. The blood pressure measurement control unit 608 causes the second blood pressure value storage unit 612 to store the calculated blood pressure value in association with time information. The blood pressure measurement control unit 608 can calculate a pulse rate while simultaneously calculating the blood pressure value. When the calculation of the blood pressure value is completed, the blood pressure measurement control unit 608 stops the pump 53 via the pump drive circuit 56 and brings the valve 54 into an open state via the valve drive circuit 57. Thus, air is exhausted from the pressing cuff 51.

The calibration unit 609 calibrates the blood pressure calculation formula, based on the pulse transit time calculated by the pulse transit time calculation unit 604 and on the blood pressure value calculated by the blood pressure measurement control unit 608. The correlation between the pulse transit time and blood pressure values varies from individual to individual. Additionally, the correlation also varies depending on the state in which the blood pressure measurement device 10 is attached to the upper arm 70 of the user. For example, even within an identical user, the correlation varies between positioning of the blood pressure measurement device 10 closer to the shoulder and positioning of the blood pressure measurement device 10 closer to the elbow. To reflect such a variation in correlation, the blood pressure calculation formula is calibrated. The calibration of the blood pressure calculation formula is performed, for example, when the blood pressure measurement device 10 is attached to the user. The calibration unit 609 obtains a plurality of sets of a measurement result for the pulse transit time and a measurement result for the blood pressure to determine parameters A₁ and A₂ based on the plurality of sets of the measurement result for the pulse transit time and the measurement result for the blood pressure. In order to determine the parameters A₁ and A₂, the calibration unit 609 uses a fitting method, for example, a least squares method or a maximum likelihood method.

Also, the present embodiment describes an example in which all the functions of the blood pressure measurement device 10 are realized by a general-purpose processor. However, some or all of the functions may be implemented by one or more dedicated processors.

### OPERATION EXAMPLE

Calibration of blood pressure calculation formula used in blood pressure measurement based on pulse transit time

Once the blood pressure measurement device 10 is attached to the user, first, the calibration of the blood pressure calculation formula is performed. Assuming that N is the number of the parameters included in the blood pressure calculation formula, N or more sets of a measurement value for the pulse transit time and a measurement value for the blood pressure are required. The blood pressure calculation Formula (1) described above includes two parameters A₁ and A₂. In this case, for example, the control unit 501 acquires a set of a measurement value for the pulse transit time and a measurement value for the blood pressure while the user is at rest, subsequently makes the user exercise, and acquires a set of a measurement value for the pulse transit time and a measurement value for the blood pressure after the exercise. Thus, two sets of the measurement value for the pulse transit time and the measurement value for the blood pressure are acquired. The control unit 501 operates as the calibration unit 609, and determines the parameters A₁ and A₂ based on the two sets of the measurement value for the pulse transit time and the measurement value for the blood pressure acquired. After the calibration is complete, blood pressure measurement based on the pulse transit time can be performed.

### Blood Pressure Measurement Based on Pulse Transit Time

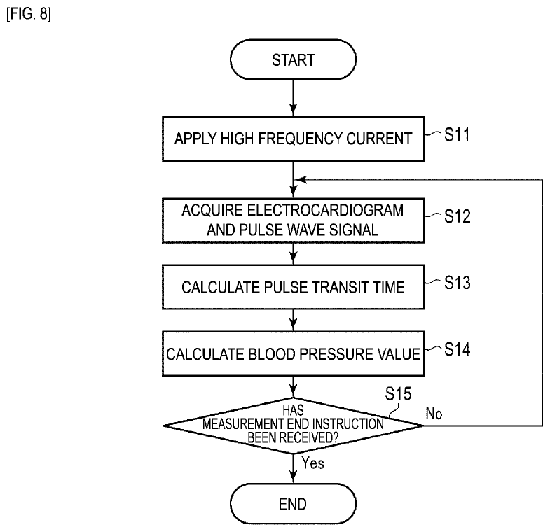

FIG. 8 illustrates an operation flow of the blood pressure measurement device 10 when performing blood pressure measurement based on the pulse transit time. The control unit 501 initiates blood pressure measurement based on the pulse transit time, for example, in response to the user instructing, through the operation unit 507, the initiation of blood pressure measurement based on the pulse transit time. Additionally, the control unit 501 may also initiate blood pressure measurement based on the pulse transit time in response to the completion of calibration of the blood pressure calculation formula.

In step S11 in FIG. 8, the control unit 501 operates as the current source control unit 601 to drive the current source 35. Accordingly, an alternating current is applied between the current electrodes 31 and 32.

In step S12, the control unit 501 acquires an electrocardiogram and a pulse wave signal at the same time. Specifically, the control unit 501 operates as the electrocardiogram generation unit 602 and acquires, as an electrocardiogram, a time series of potential difference signals output from the detection circuit 380. Furthermore, the control unit 501 operates as the pulse wave signal generation unit 603 and acquires, as a pulse wave signal, a time series of potential difference signals output from the detection circuit 370.

In step S13, the control unit 501 operates as the pulse transit time calculation unit 604 and calculates, as the pulse transit time, a time difference between the R wave peak point in the electrocardiogram and the rising point in the pulse wave signal. In step S14, the control unit 501 operates as the blood pressure value calculation unit 605 and calculates a blood pressure value from the pulse transit time calculated in step S13 using the blood pressure calculation Formula (1) described above. The control unit 501 stores the calculated blood pressure value in the storage unit 505 in association with time information.

In step S15, the control unit 501 determines whether the user has instructed, through the operation unit 507, the termination of the blood pressure measurement based on the pulse transit time. The processing from step S12 to step S14 is repeated until the user instructs the termination of the blood pressure measurement based on the pulse transit time. Thus, the blood pressure value for each beat is recorded. When the user instructs the termination of the blood pressure measurement based on the pulse transit time, the control unit 501 operates as the current source control unit 601 to stop the current source 35. Thus, the blood pressure measurement based on the pulse transit time is terminated.

With the blood pressure measurement based on the pulse transit time, the blood pressure can be continuously measured over an extended period of time with a reduced physical burden on the user.

### Blood Pressure Measurement Using Oscillometric Method

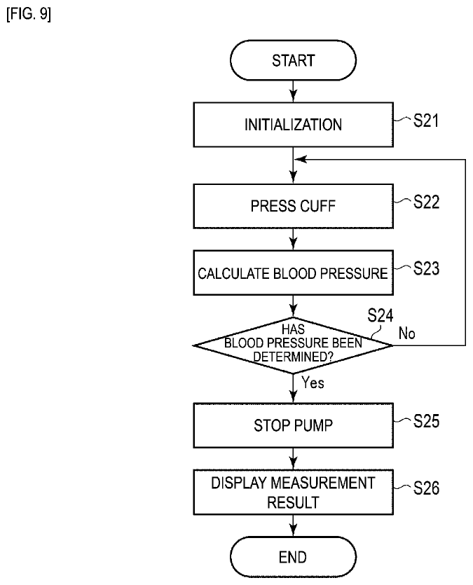

FIG. 9 illustrates an operation flow of the blood pressure measurement device 10 when performing blood pressure measurement using the oscillometric method. In the blood pressure measurement using the oscillometric method, the pressing cuff 51 is gradually pressurized and then depressurized. In such a pressurization or depressurization process, the pulse transit time fails to be measured correctly. Thus, during the execution of the blood pressure measurement using the oscillometric method, the blood pressure measurement based on the pulse transit time illustrated in FIG. 8 may be temporarily stopped.

In response to the user having instructed, through the operation unit 507, execution of the blood pressure measurement using the oscillometric method, the control unit 501 initiates the blood pressure measurement.

In step S21 in FIG. 9, the control unit 501 operates as the blood pressure measurement control unit 608 to perform initialization for the blood pressure measurement using the oscillometric method. For example, the control unit 501 initializes a processing memory area. Then, the control unit 501 stops the pump 53 via the pump drive circuit 56, and brings the valve 54 into the open state via the valve drive circuit 57. Accordingly, the air in the pressing cuff 51 is discharged. The control unit 501 sets an output value at the moment from the pressure sensor 52 as a reference value.

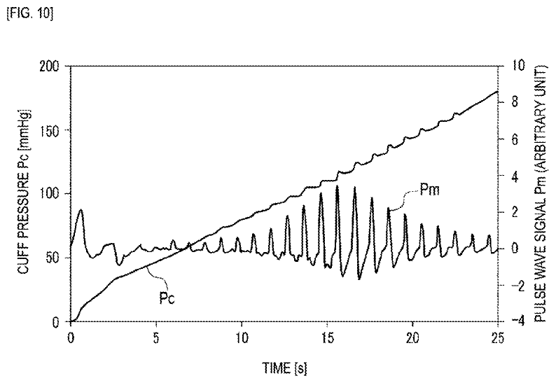

In step S22, the control unit 501 operates as the blood pressure measurement control unit 608 to perform control for pressurizing the pressing cuff 51. For example, the control unit 501 brings the valve 54 into the closed state via the valve drive circuit 57 and drives the pump 53 via the pump drive circuit 56. Accordingly, air is fed to the pressing cuff 51 to inflate the pressing cuff 51, and a cuff pressure Pc gradually increases as illustrated in FIG. 10. The control unit 501 monitors the cuff pressure Pc using the pressure sensor 52 and acquires a pulse wave signal Pm representing a fluctuation component of an arterial volume.

In step S23, the control unit 501 operates as blood pressure measurement control unit 608 and attempts to calculate the blood pressure value (including the SBP and the DBP) based on the pulse wave signal Pm acquired at this point in time. In a case where the blood pressure value fails to be calculated yet due to lack of data at this point in time (No in step S24), the processing in steps S22 and S23 is repeated as long as the cuff pressure Pc does not reach an upper pressure limit. The upper limit pressure is predetermined from the viewpoint of safety. The upper pressure limit is set to 300 mmHg, for example.

In a case where the blood pressure value can be calculated (Yes in step S24), then the processing proceeds to step S25. In step S25, the control unit 501 operates as the blood pressure measurement control unit 608, stops the pump 53 via the pump drive circuit 56, and brings the valve 54 into the open state via the valve drive circuit 57. Accordingly, the air in the pressing cuff 51 is discharged.

In step S26, the control unit 501 displays blood pressure measurement results on the display unit 506 and records the blood pressure measurement results in the storage unit 505.

Note that the processing procedure illustrated in FIG. 8 or FIG. 9 is illustrative, and the processing sequence can be changed as appropriate. The contents of each type of processing can also be changed as appropriate. For example, in the blood pressure measurement using the oscillometric method, the calculation of blood pressure values may be performed in the depressurization process in which air is discharged from the pressing cuff 51.

### Effects

As described above, in the present embodiment, the ECG sensor, the pulse wave sensor, the pressing cuff 51, and the like are provided in the belt unit 20. Thus, in order to measure the pulse transit time or the blood pressure, the user may simply wind the belt unit 20 around the upper arm 70. Thus, the blood pressure measurement device 10 can be easily attached to the user. One device needs to be attached to the user, thus reducing the attachment burden on the user.

Furthermore, the ECG sensor and the pulse wave sensor share a detection electrodes 33 and 34 and the potential difference signal detection unit 36 (for example, the instrumentation amplifier 360). This reduces a region of the inner circumferential surface of the belt unit 20 required for disposing the electrodes, enabling a reduction in the size of the blood pressure measurement device 10. The miniaturization of the blood pressure measurement device 10 contributes to reducing the attachment burden. Furthermore, part costs can be reduced because there is no need to prepare a detection electrode and a potential difference signal detection unit for each of the ECG sensor and the pulse wave sensor.

### MODIFIED EXAMPLES

Note that the present invention is not limited to the embodiments described above.

In one embodiment, the pressing cuff 51 may be used to adjust a contact state between the upper arm 70 and the current electrodes 31 and 32 and the detection electrodes 33 and 34.

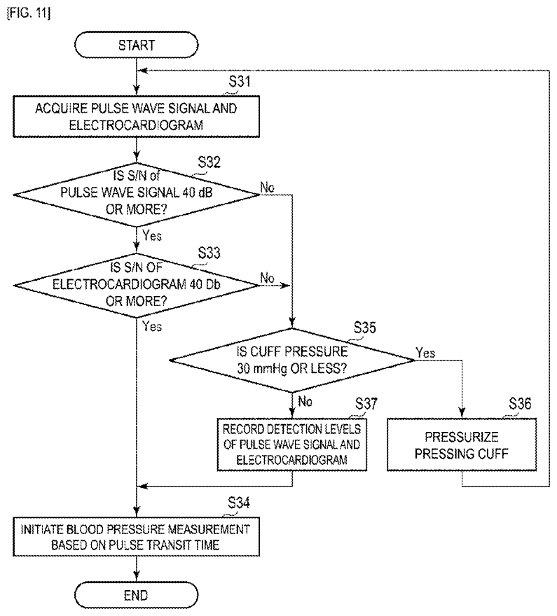

FIG. 11 illustrates an operation flow of the blood pressure measurement device 10 when adjusting the contact state between the electrodes and the upper arm 70. In step S31 in FIG. 11, the control unit 501 acquires a pulse wave signal and an electrocardiogram. Processing in step S31 is similar to the processing described with respect to steps S11 and S12 of FIG. 8, and thus the description of the processing is omitted.

In step S32, the control unit 501 determines whether the signal-to-noise ratio of the pulse wave signal acquired in step S31 is greater than or equal to a first threshold. The first threshold is, for example, 40 dB. In a case where the signal-to-noise ratio of the pulse wave signal is greater than or equal to the first threshold, the processing proceeds to step S33, and in a case where the signal-to-noise ratio of the pulse wave signal is less than the first threshold, the processing proceeds to step S35.

In step S33, the control unit 501 determines whether the signal-to-noise ratio of the electrocardiogram acquired in step S31 is greater than or equal to a second threshold. The second threshold is, for example, 40 dB. Note that the second threshold may be different from the first threshold. In a case where the signal-to-noise ratio of the electrocardiogram is greater than or equal to the second threshold, the processing proceeds to step S34, and in a case where the signal-to-noise ratio of the electrocardiogram is less than the second threshold, the processing proceeds to step S35.

In step S35, the control unit 501 determines whether the cuff pressure is less than or equal to the third threshold. The third threshold is, for example, 30 mmHg. In an initial state, the cuff pressure is equal to the reference value (0 mmHg). In a case where the cuff pressure is less than or equal to the third threshold, the processing proceeds to step S36. In step S36, the control unit 501 drives the pump 53 via the pump drive circuit 56 to increase the cuff pressure. For example, the cuff pressure is increased by 10 mmHg. The processing then returns to step S31.

In a case where the cuff pressure exceeds the third threshold in step S35, the processing proceeds to step S37. In step S37, the control unit 501 causes the storage unit 505 to store the detection levels of the pulse wave signal and the electrocardiogram acquired at the cuff pressure at the moment. The processing then proceeds to step S34.

In step S34, the control unit 501 initiates blood pressure measurement illustrated in FIG. 8 based on the pulse transit time.

By adjusting the contact state between the upper arm and the electrodes in this manner, a pulse wave signal and an electrocardiogram having the desired signal-to-noise ratio can be acquired. As a result, the measurement accuracy for the pulse transit time is improved.

In an embodiment, a plurality of detection electrodes 33 or a plurality of detection electrodes 34 may be provided in the belt unit 20.

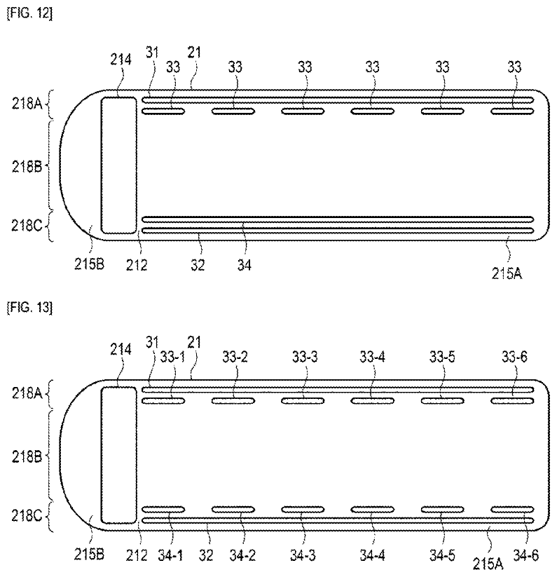

FIG. 12 illustrates the appearance of a blood pressure measurement device according to an embodiment. In the blood pressure measurement device illustrated in FIG. 12, six detection electrodes 33 and one detection electrode 34 are disposed on the inner circumferential surface 212 of the belt 21. The detection electrodes 33 are arranged at regular intervals in the longitudinal direction of the belt 21. In this arrangement, for example, for a user expected to have the thinnest upper arm, four of the six detection electrodes 33 contact the upper arm 70 in the attachment state, and the remaining two detection electrodes 33 contact the outer circumferential surface 211 of the belt 21. For a user expected to have the thickest upper arm, all the six detection electrodes 33 contact the upper arm 70 in the attachment state.

FIG. 13 illustrates the appearance of a blood pressure measurement device according to an embodiment. In the blood pressure measurement device illustrated in FIG. 13, six detection electrodes 33 and six detection electrodes 34 are disposed on the inner circumferential surface 212 of the belt 21. The detection electrodes 33 are arranged at regular intervals in the longitudinal direction of the belt 21, and the detection electrodes 34 are arranged at regular intervals in the longitudinal direction of the belt 21. In FIG. 13, the reference numerals include branch numbers to differentiate individual detection electrodes 33 and 34. Detection electrodes 33-1, 33-2, 33-3, 33-4, 33-5, and 33-6 respectively face detection electrodes 34-1, 34-2, 34-3, 34-4, 34-5, and 34-6 in the width direction of the belt 21.

The potential of the detection electrode 33 illustrated in FIG. 3 corresponds to the average of the potentials of the detection electrodes 33-1 to 33-6 illustrated in FIG. 13. Similarly, the potential of the detection electrode 34 illustrated in FIG. 3 corresponds to the average of the potentials of the detection electrodes 34-1 to 34-6 illustrated in FIG. 13. Thus, the signal-to-noise ratio can be improved by selecting one appropriate detection electrode 33 from the detection electrode 33-1 to 33-6, selecting one appropriate detection electrode 34 from the detection electrode 34-1 to 34-6, and acquiring a pulse wave signal and an electrocardiogram based on the potential difference between the selected detection electrodes 33 and 34.

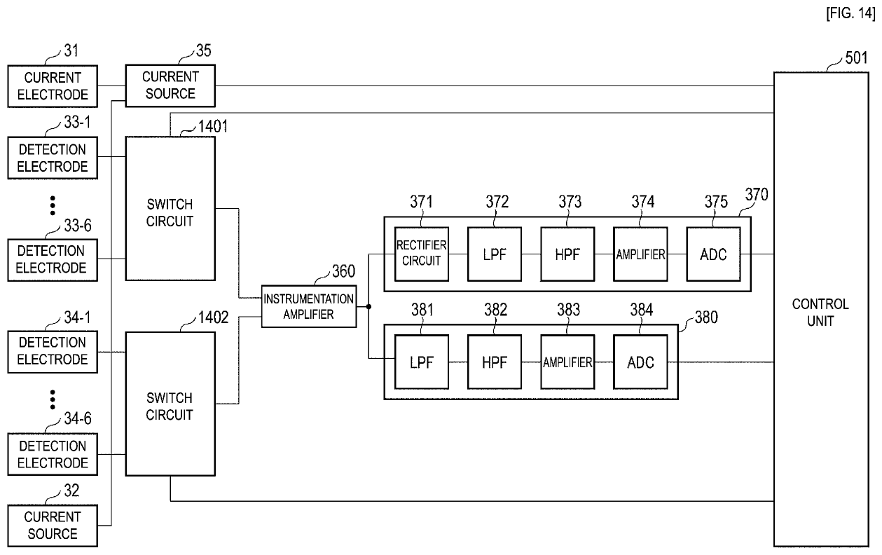

FIG. 14 illustrates a hardware configuration of a control system of the blood pressure measurement device illustrated in FIG. 13. In FIG. 14, some components have been omitted, such as the components involved in the blood pressure measurement using the oscillometric method. Additionally, in FIG. 14, the same components as those illustrated in FIG. 5 are denoted by the same reference numerals, and detailed descriptions of these components are omitted.

The blood pressure measurement device illustrated in FIG. 14 includes a switch circuit 1401 and a switch circuit 1402 in addition to the components illustrated in FIG. 5. The switch circuit 1401 is provided between the instrumentation amplifier 360 and six detection electrodes 33 and switches, among the six detection electrodes 33, the detection electrode 33 to be connected to the instrumentation amplifier 360. The switch circuit 1401 connects, to the instrumentation amplifier 360, the detection electrode 33 designated by a switch signal received from the control unit 501. The switch circuit 1402 is provided between the instrumentation amplifier 360 and six detection electrodes 34 and switches, among the six detection electrodes 34, the detection electrode 34 to be connected to the instrumentation amplifier 360. The switch circuit 1402 connects, to the instrumentation amplifier 360, the detection electrode 34 designated by a switch signal received from the control unit 501.

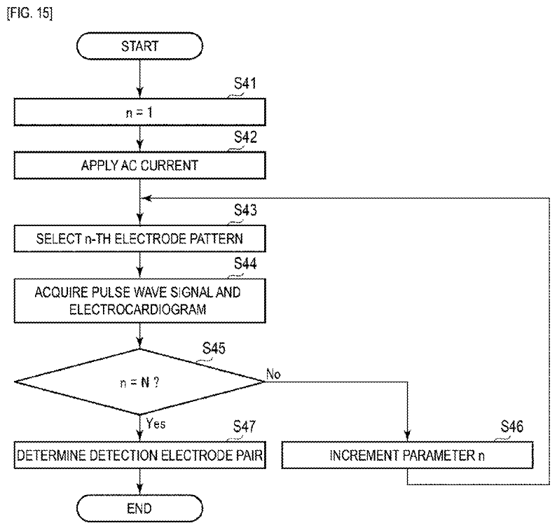

FIG. 15 illustrates an operation flow of the blood pressure measurement device 10 illustrated in FIG. 14 when selecting an electrode pair used to acquire an electrocardiogram and a pulse wave signal. The operation flow illustrated in FIG. 15 is initiated, for example, in response to attachment of the blood pressure measurement device 10 to the user. The operation flow may also be initiated in response to an instruction from the user or each time a period of time elapses.

Here, N electrode patterns are set as candidates for the detection electrode pair used to acquire an electrocardiogram and a pulse wave signal. In one example, six electrode patterns are set including a pair of the detection electrode 33-1 and the detection electrode 34-1, a pair of the detection electrode 33-2 and the detection electrode 34-2, . . . and a pair of the detection electrode 33-6 and the detection electrode 34-6. In other examples, all detection electrode pairs formed by the six detection electrodes 33 and the six detection electrodes 34 may be set as electrode patterns. In this example, 36 electrode patterns are set.

In step S41 in FIG. 15, the control unit 501 initializes a parameter n. For example, the control unit 501 sets the parameter n to 1. In step S42, the control unit 501 operates as the current source control unit 601 to drive the current source 35. Accordingly, an alternating current is applied between the current electrodes 31 and 32.

In step S43, the control unit 501 selects the n-th electrode pattern. For example, the control unit 501 provides the switch circuit 1401 with a switch signal designating the detection electrode 33 corresponding to the n-th electrode pattern and provides the switch circuit 1402 with a switch signal designating the detection electrode 34 corresponding to the n-th electrode pattern. Thus, the detection electrodes 33 and 34 corresponding to the n-th electrode pattern is connected to the instrumentation amplifier 360.

In step S44, the control unit 501 acquires a pulse wave signal and an electrocardiogram based on a potential difference between the detection electrodes 33 and 34. Specifically, the control unit 501 operates as the pulse wave signal generation unit 603 and acquires, as a pulse wave signal, a time series of potential difference signals output from the detection circuit 370. Furthermore, the control unit 501 operates as the electrocardiogram generation unit 602 and acquires, as an electrocardiogram, a time series of potential difference signals output from the detection circuit 380. The control unit 501 causes the storage unit 505 to store the acquired electrocardiogram and pulse wave signal in association with the parameter n.

In step S45, the control unit 501 determines whether the parameter n is equal to N or not. In a case where the parameter n is not equal to N, the processing proceeds to step S46, and the control unit 501 increments the parameter n by 1. The processing then returns to step S43.

In step S45, in a case where the parameter n is equal to N, the processing proceeds to step S47. In this case, an electrocardiogram and a pulse wave signal are acquired for each of the N electrode patterns.

In step S47, the control unit 501 acts as an electrode selection unit and applies a predetermined selection criterion to the N electrode patterns to select one of the N electrode patterns as a detection electrode pair used to acquire an electrocardiogram and a pulse wave signal. A selection criterion may be, for example, the condition that the signal-to-noise ratio of the electrocardiogram exceeds the first threshold and that the signal-to-noise ratio of the pulse wave signal exceeds the second threshold. The first threshold may be the same value as the second threshold and may be a value different from the second threshold. According to the selection criterion, an electrode pattern is selected that provides an electrocardiogram having a signal-to-noise ratio exceeding the first threshold and a pulse wave signal having a signal-to-noise ratio exceeding the second threshold. A plurality of electrode patterns may meet the selection criterion described above. Thus, the selection criterion may further include a condition for selecting one electrode pattern. The further condition is, for example, the condition that the electrocardiogram has the greatest signal-to-noise ratio.

Selecting the detection electrode pair in this way results in the acquisition of an electrocardiogram and a pulse wave signal with a higher signal-to-noise ratio. As a result, the pulse transit time can be accurately measured.

Note that the detection electrode pair used to acquire a pulse wave signal may be different from the detection electrode pair used to acquire an electrocardiogram. As an example, the detection electrodes 33-3 and 34-3 are used to acquire a pulse wave signal, and the detection electrode 33-1 and 33-3 is used to acquire an electrocardiogram. In this case, two instrumentation amplifiers are provided.

In an embodiment, a plurality of current electrodes 31 or a plurality of current electrodes 32 may be provided in the belt unit 20.

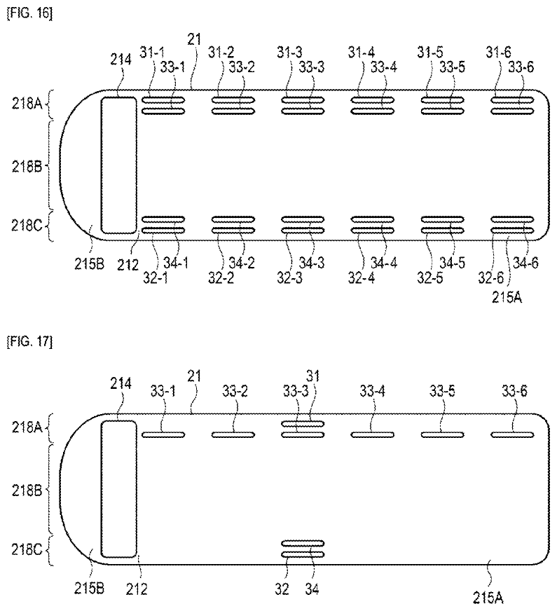

FIG. 16 illustrates the appearance of a blood pressure measurement device according to an embodiment. In the blood pressure measurement device illustrated in FIG. 16, the six current electrodes 31, the six current electrodes 32, the six detection electrodes 33, and the six detection electrodes 34 are disposed on the inner circumferential surface 212 of the belt 21. The current electrodes 31 are arranged at regular intervals in the longitudinal direction of the belt 21, the current electrodes 32 are arranged at regular intervals in the longitudinal direction of the belt 21, the detection electrodes 33 are arranged at regular intervals in the longitudinal direction of the belt 21, and the detection electrodes 34 are arranged at regular intervals in the longitudinal direction of the belt 21. In FIG. 16, the reference numerals include branch numbers to differentiate individual current electrodes 31 and 32 and detection electrodes 33 and 34. The current electrode 31-m, the detection electrode 33-m, the detection electrode 34-m, and the current electrode 32-m are aligned in this order in the width direction of the belt 21. Here, m is an integer from 1 to 6.

In the blood pressure measurement device illustrated in FIG. 16, the current electrodes 31 and 32 used for electric connection are selected depending on the detection electrodes 33 and 34 used to acquire a pulse wave signal. For example, in a case where the detection electrode 33-3 and 34-3 are used to acquire a pulse wave signal, a high frequency current is applied between the current electrodes 31-3 and 32-3.

In an embodiment, an electrocardiogram may be acquired using two detection electrodes selected from among the plurality of detection electrodes disposed in the longitudinal direction of the belt 21.

FIG. 17 illustrates the appearance of a blood pressure measurement device according to an embodiment. In the blood pressure measurement device illustrated in FIG. 17, one current electrode 31, one current electrode 32, six detection electrodes 33 and one detection electrode 34 are disposed on the inner circumferential surface 212 of the belt 21. The detection electrodes 33 are arranged in the longitudinal direction of the belt 21. In FIG. 17, the reference numerals include branch numbers to differentiate individual detection electrodes 33. In this example, the detection electrode 34 faces the detection electrode 33-3 in the width direction of the belt 21 and has an identical length to the detection electrode 33-3 (dimension in the longitudinal direction of the belt 21).

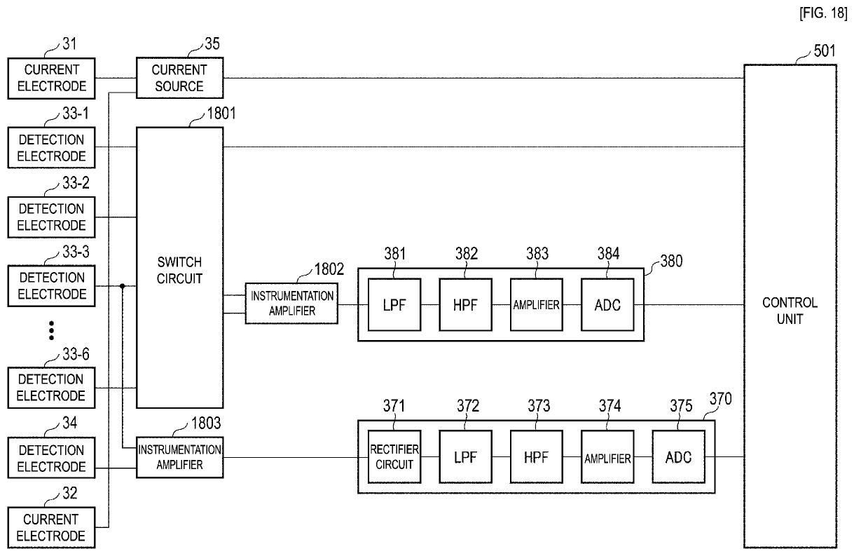

FIG. 18 illustrates an example of a hardware configuration of a control system of the blood pressure measurement device illustrated in FIG. 17. In FIG. 18, some components are omitted, such as the components involved in the blood pressure measurement using the oscillometric method. Additionally, in FIG. 18, identical components to the components illustrated in FIG. 5 are denoted by the same reference numerals, and detailed descriptions of these components are omitted.

The blood pressure measurement device illustrated in FIG. 18 includes the current source 35, a switch circuit 1801, an instrumentation amplifier 1802, an instrumentation amplifier 1803, the detection circuit 370, the detection circuit 380, and the control unit 501 in addition to the current electrode 31, the current electrode 32, the detection electrode 33-1, . . . and 33-6, and the detection electrode 34.

The switch circuit 1801 is provided between the instrumentation amplifier 1802 and the detection electrodes 33-1 to 33-6. The switch circuit 1801 connects two of the detection electrodes 33-1 to 33-6 to the instrumentation amplifier 1802 according to the switch signal received from the control unit 501. The instrumentation amplifier 1802 outputs, to the detection circuit 380, a potential difference signal between the two detection electrodes 33 connected to the input terminal.

The detection electrode 33-3 and the detection electrode 34 are connected to input terminals of the instrumentation amplifier 1803. The instrumentation amplifier 1803 outputs, to the detection circuit 370, a potential difference signal between the detection electrode 33-3 and the detection electrode 34.

A portion of the blood pressure measurement device involved in the measurement of the pulse transit time may be implemented as a single device. In one embodiment, a pulse transit time measurement device is provided that includes the belt unit 20, the current electrodes 31 and 32, the detection electrodes 33 and 34, the current source 35, the potential difference signal detection unit 36, the pulse wave signal acquisition unit 37, the electrocardiogram acquisition unit 38, and the pulse transit time calculation unit 39.

The blood pressure measurement device 10 need not include the second blood pressure measurement unit 50. In embodiments in which the blood pressure measurement device 10 does not include the second blood pressure measurement unit 50, a blood pressure value obtained by measurement with another blood pressure monitor needs to be input to the blood pressure measurement device 10 for calibration of the blood pressure calculation formula.

The target measurement site is not limited to the upper arm and may be another site such as the wrist, thigh, or ankle. The target measurement site can be a part of any of the limbs.

In short, the present invention is not limited to the embodiment described above as is, and the components can be modified and embodied within a range that does not depart from the gist in a stage of implementation. Further, various inventions can be formed by appropriately combining a plurality of components disclosed in the embodiment described above. For example, some components may be omitted from the all components illustrated in the embodiment. Furthermore, the components of different embodiments may be combined appropriately.

### REFERENCE SIGNS LIST

10 Blood pressure measurement device
20 Belt unit
21 Belt
22 Body
30 First blood pressure measurement unit
31, 32 Current electrode
33, 34 Detection electrode
35 Current source
36 Potential difference signal detection unit
37 Pulse wave signal acquisition unit
38 Electrocardiogram acquisition unit
39 Pulse transit time calculation unit
40 Blood pressure value calculation unit
50 Second blood pressure measurement unit
51 Pressing cuff
52 Pressure sensor
53 Pump
54 Valve
55 Oscillation circuit
56 Pump drive circuit
57 Valve drive circuit
58, 59 Pipe
210A Inner cloth
210B Outer cloth
211 Outer circumferential surface
212 Inner circumferential surface
213 Loop surface
214 Hook surface
360 Instrumentation amplifier
370 Detection circuit
371 Rectifier circuit
372 LPF
373 HPF
374 Amplifier
375 ADC
380 Detection circuit
381 LPF
382 HPF
383 Amplifier
384 ADC
501 Control unit
502 CPU
503 RAM
504 ROM
505 Storage unit
506 Display unit
507 Operation unit
508 Communication unit
509 Battery
601 Current source control unit
602 Electrocardiogram generation unit
603 Pulse wave signal generation unit
604 Pulse transit time calculation unit
605 Blood pressure value calculation unit
606 Instruction input unit
607 Display control unit
608 Blood pressure measurement control unit
609 Calibration unit
611 First blood pressure value storage unit
612 Second blood pressure value storage unit
1401, 1402, 1801 Switch circuit
1802, 1803 Instrumentation amplifier

1. A pulse transit time measurement device comprising:
one belt unit wound around a target measurement site corresponding to a part of any of limbs of a user;
an electrode group provided in the belt unit and including a first electrode, a second electrode, a third electrode, and a fourth electrode;
a current source applying an alternating current between the first electrode and the second electrode;
a potential difference signal detection unit detecting a potential difference signal between the third electrode and the fourth electrode;
an electrocardiogram acquisition unit acquiring, based on the potential difference signal, an electrocardiogram corresponding to a waveform signal representative of an electrical activity of a heart of the user;
a pulse wave signal acquisition unit acquiring, based on the potential difference signal, as a pulse wave signal, a waveform signal representative of an electrical impedance in the target measurement site of the user; and
a pulse transit time calculation unit calculating a pulse transit time based on the electrocardiogram and the pulse wave signal.

2. The pulse transit time measurement device according to claim 1, wherein the electrode group includes a plurality of the third electrodes, and the plurality of third electrodes are arranged in one direction, and
the pulse transit time measurement device further comprises a first switch circuit switching, among the plurality of third electrodes, to the third electrode to be connected to the potential difference signal detection unit.

3. The pulse transit time measurement device according to claim 2, wherein the electrode group includes a plurality of the fourth electrodes, and the plurality of fourth electrodes are arranged in the one direction, and
the pulse transit time measurement device further comprises a second switch circuit switching, among the plurality of fourth electrodes, to the fourth electrode to be connected to the potential difference signal detection unit.

4. A pulse transit time measurement device comprising:
a belt unit wound around a target measurement site of a user;
an electrode group wound around the belt unit and including a first electrode, a second electrode, a plurality of third electrodes arranged in a row, and a fourth electrode;
a current source applying an alternating current between the first electrode and the second electrode;
a first potential difference signal detection unit detecting a first potential difference signal corresponding to a potential difference signal between one of the plurality of third electrodes and the fourth electrode;
a pulse wave signal acquisition unit acquiring, based on the first potential difference signal, as a pulse wave signal, a waveform signal representative of an electrical impedance in the target measurement site of the user;
a second potential difference signal detection unit detecting a second potential difference signal corresponding to a potential difference signal between two of the third electrodes selected from among the plurality of third electrodes;
an electrocardiogram acquisition unit acquiring, based on the second potential difference signal, an electrocardiogram corresponding to a waveform signal representative of an electrical activity of a heart of the user; and
a pulse transit time calculation unit calculating a pulse transit time based on the electrocardiogram and the pulse signal.

5. A blood pressure measurement device comprising:
the pulse transit time measurement device according to claim 1; and
a first blood pressure value calculation unit calculating a first blood pressure value based on the pulse transit time that is calculated.

6. The blood pressure measurement device according to claim 5, further comprising:
a pressing cuff provided in the belt unit;
a fluid supply unit supplying a fluid to the pressing cuff;
a pressure sensor detecting a pressure in the pressing cuff; and
a second blood pressure value calculation unit calculating a second blood pressure value based on an output of the pressure sensor.
